(19) United States
(12) Patent Application Publication
(10) Pub. No.: US 2009/0318379 A1
(43) Pub. Date: Dec. 24, 2009

(54) Statins for the Treatment of Viral Influenza Infections

(76) Inventor: Patrick T. Prendergast, New South Wales (AU)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 12/226,960

(22) PCT Filed: May 4, 2007
(86) PCT No.: PCT/IE2007/000052

§ 371 Date: Dec. 1, 2008

Related U.S. Application Data
(60) Provisional application No. 60/811,007, filed on Jun. 5, 2006.
(60) Provisional application No. 60/835,824, filed on Aug. 4, 2006.

(30) Foreign Application Priority Data
May 4, 2006 (GB) 0608792.8

(51) Int. Cl. A61K 31/70; A61K 31/35; A61K 31/225; A61K 31/404; A61K 31/44; A61K 31/40; A61P 31/16

(52) U.S. Cl. 514/43; 514/460; 514/548; 514/415; 514/277; 514/423; 514/459

(57) ABSTRACT

The present invention relates to methods for the treatment of viral influenza using statins, or pharmaceutically effective salts, pro-drugs or metabolites thereof. In particular it is disclosed that statin hydroxyl acid salts including, but not limited to, mevastatin, lovastatin, pravastatin and simvastatin, are effective in conferring resistance to infection with influenza type A viral challenge.

### FIELD OF INVENTION

The present invention provides a novel method for the treatment of viral influenza infection. In particular, there is provided a method for the prophylaxis and/or treatment of influenza which comprises the administration to a subject of a therapeutically effective amount of a statin or statin-like compound, such as mevastatin, lovastatin, pravastatin or simvastatin.

### BACKGROUND TO THE INVENTION

Influenza viruses are orthomyxoviruses, which fall into three subtypes; A, B and C. Influenza A and B virus particles contain a genome of negative sense, single-stranded RNA divided into 8 linear segments. Co-infection of a single host with two different influenza viruses may result in the generation of ‘reassortant’ progeny viruses having a new combination of genome segments, derived from each of the parental viruses.

Influenza A viruses have been responsible for four recent pandemics of severe human respiratory illness. Influenza A viruses can be divided into subtypes according to their surface proteins, hemagglutinin (HA or H) and neuraminidase (NA or N). There are 14 known H subtypes and 9 known N subtypes. Only three H subtypes (H1, H2 and H3) and two N subtypes (N1 and N2) have been reported as commonly circulating in humans.

Seasonal influenza epidemics in humans are associated with amino acid changes in antigenic sites in the hemagglutinin and neuraminidase proteins, in a process termed ‘antigenic drift’. Major pandemics are associated with the introduction of new hemagglutinin and neuraminidase genes from animal-derived influenza viruses, by reassortment, into the genetic background of a currently circulating human virus—called ‘antigenic shift’.

Several factors contribute to the epidemiological success of influenza virus. Firstly, it is spread easily from person to person by aerosol (also referred to as droplet infection). Secondly, small changes in influenza virus antigens are frequent (antigenic drift) so that the virus readily escapes protective immunity induced by a previous exposure to a different variant of the virus. Third, new strains of influenza virus can be easily generated by reassortment or mixing of genetic material between different strains (antigenic shift). In the case of influenza A virus, such mixing can occur between subtypes or strains that affect different species.

Despite intensive efforts, there is still no effective therapy for influenza virus infection. The effectiveness of existing vaccines are limited due to the occurrence of antigenic shift and antigenic drift. Although current vaccines, which are based upon inactivated virus, are able to prevent illness in approximately 70-80% of healthy individuals under age 65, this percentage is far lower in the elderly or immunocompromised subjects. In addition, the expense and potential side effects associated with vaccine administration make this approach less than optimal.

At present, there are two classes of drugs commercially available for the prevention and treatment of influenza virus infections in humans; M2 ion channel blockers and Neuraminidase inhibitors. Amantadine and Rimantadine function by blocking the ion channel activity of the viral M2 protein which is mainly required during virus entry in the early phase of the replication life cycle. Both treatments are highly effective in treating influenza A but cause significant side effects on the central nervous system, liver and kidneys. Sensitive influenza strains rapidly develop resistance in vitro and in vivo.

Oseltamivir and Zanamivir block the action of neuraminidase to prevent the release of newly formed virus from infected cells and spread within the host. Both drugs efficiently inhibited influenza viruses in clinical studies, however escape from the selective pressures of neuraminidase inhibitors has been observed in cell culture and in patients.

Many of the current anti-viral therapies are directed towards targeting viral components and are therefore prone to compensatory viral escape mechanisms.

Treatments aimed at manipulating the host to interfere with viral replication, either by enhancing antiviral responses or by inhibiting proviral activities within the host cell have greater potential to control influenza without selective pressure on the virus itself to mutate in a compensatory manner are desirable. The possibility for combination therapy targeting virus and host at the same time, to minimise the opportunity for the virus to acquire resistance is also particularly appealing.

Ribavirin is a broad-spectrum anti-viral agent based on a purine nucleoside analogue and is the standard treatment regimen for hepatitis C. Ribavirin is known to be active against various RNA viruses by inducing lethal mutagenesis of the viral RNA genome and is known to show anti-viral activity against animal cornaviruses. Although Ribavirin has a marked anti-viral activity against a number of viruses, it is not acknowledged as a medicament for influenza infections. In addition, the considerable toxicity associated with Ribavirin limits its utility as a medicament.

There therefore remains a need for the development of effective therapies for the treatment and prevention of influenza infection.

Statins are compounds that are known to have a lowering effect on levels of LDL-cholesterol in the human blood. Statins inhibit the hydroxymethylglutaryl coenzyme A (HMG-CoA) reductase, the rate-determining step in the cholesterol biosynthesis.

Currently available statins include lovastatin, simvastatin, pravastatin, fluvastatin, cerivastatin and atorvastatin. Lovastatin (disclosed in U.S. Pat. No. 4,231,938) and simvastatin (ZOCOR; disclosed in U.S. Pat. No. 4,444,784 and WO 00/53566) are administered in the lactone form. After absorption, the lactone ring is opened in the liver by chemical or enzymatic hydrolysis, and the active hydroxy acid is generated.

Pravastatin (PRAVACHOL; disclosed in U.S. Pat. No. 4,346,227) is administered as the sodium salt. Fluvastatin (LESCOL; disclosed in U.S. Pat. No. 4,739,073) and cerivastatin (disclosed in U.S. Pat. Nos. 5,006,530 and 5,177,080), also administered as the sodium salt, are entirely synthetic compounds that are in part structurally distinct from the fungal derivatives of this class that contain a hexahydronaphthalene ring. Atorvastatin and two new “superstatins”, called rosuvastatin and pitavastatin, are administered as calcium salts.

International patent application No WO 00/47196 relates to a method for treating a mammal suffering from hepatitis C, HIV, hepatitis B, hepatitis G, or hepatitis H by administering to the patient at least one statin or statin-like compound.

However, WO 00/47196 did not consider the utility of said statin compounds in the prophylaxis of such viral infections, nor in viral infections other than those listed therein.

The present inventor has surprisingly found that statins or precursors thereof, or statin-like compounds can act in a prophylactic manner in order to prevent influenza infection with type A influenza infection. In particular, it has been surprisingly and unexpectedly found that statin hydroxyl acid salts, the active metabolite of the prodrug compounds known in the art as statins, which include, but are not limited to, mevastatin, lovastatin, pravastatin and simvastatin, are effective in the prophylactic treatment of a vertebrate, particularly a mammal and more particularly a human a exposed to any strain of influenza virus.

The present inventor has surprisingly found that the administration of the active metabolite of a statin prodrug (the hydroxyl acid salt) to a patient at specified dosages, by any route of administration, but preferable by nasal administration, will confer infection resistance to influenza virus challenge. Without wishing to be bound by theory, the inventor predicts that the statin metabolite compound functions to prevent influenza virus attachment and infection to the nasal/thoracic epithelial cells to which the influenza virus has specific affinity for. This therapeutic approach has utility in preventing influenza infection by strains of Influenza A which result from virus reassortment.

### SUMMARY OF THE INVENTION

According to a first aspect of the present invention there is provided a method for the treatment and/or prophylaxis of infection with Type A Influenza, the method comprising the steps of:
providing a composition comprising at least one statin or an analogue, derivative or metabolite thereof or a pharmaceutically active salt or pro-drug thereof, and
administering a therapeutically effective or prophylactically effective amount of said composition to a subject in need of treatment.

In certain embodiments, the at least one statin is a statin metabolite. In further certain embodiments, a statin prodrug is administered, this being metabolized to a statin metabolite which has utility in the methods of the present invention.

In certain embodiments, the subject is a mammal, typically a human.

In certain embodiments, the statin metabolite may be a metabolite of at least one statin hydroxy acid salt or an analogue, derivative, metabolite, prodrug or salt thereof.

In certain embodiments, the statin is lovastatin hydroxy acid sodium salt or an analogue, derivative, metabolite, prodrug or salt thereof. Lovastatin hydroxy acid sodium salt is also known as mevinolinic acid, this compound having a weight of 444.54 and a CAS number [75225-50-2].

In certain embodiments, lovastatin is defined by the following chemical structure, which is defined herein as formula I:

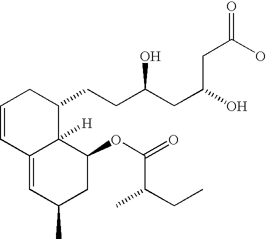

In certain embodiments, the lovastatin hydroxy acid sodium salt also known by the name Mevinolinic acid, has the structure as defined below in formula II:

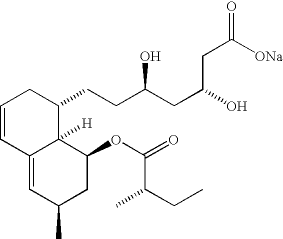

In certain further embodiments, the statin compound is Mevinolinic acid or an analogue, derivative, metabolite, prodrug or salt thereof.

As herein defined, references to a “statin” or “statins” are intended to embrace the statin in free base form, or as a pharmaceutically acceptable salt of said statin. Further, when reference is made to a “pro-drug” of a statin, it is further intended to embrace salts of the pro-drug.

In certain further embodiments, the statin pro-drug may be selected from the group consisting of, but not limited to: lovastatin, simvastatin, pravastatin, fluvastatin, cerivastatin and atorvastatin or an analogue, derivative, metabolite, prodrug or salt thereof.

In certain further embodiments, the statin pro-drug may be a pharmaceutically active salt of lovastatin, simvastatin, pravastatin, fluvastatin, cerivastatin and atorvastatin. In certain further embodiments, the statin is a hydroxyl acid salt of lovastatin, simvastatin, pravastatin, fluvastatin, cerivastatin and atorvastatin.

In certain embodiments, the statin is pravastatin hydroxy acid salt or an analogue, derivative, metabolite or a prodrug thereof.

In certain embodiments, the statin is simvastatin hydroxy acid salt an analogue, derivative, prodrug or metabolite thereof.

In certain embodiments the statin is mevastatin hydroxy acid salt or an analogue, derivative, prodrug or metabolite thereof.

In certain embodiments, the Influenza A virus is of the subtype H3. In certain further embodiments, the subtype is H3N2.

In certain embodiments, the Influenza A virus is selected from the group comprising, but not limited to, Influenza A virus of the subtype; H5, H7 or H9. In further certain embodiments the type A Influenza subtype is of the strain H5N1, H5N2, H9N2, H7N2 or H7N7.

A further aspect of the present invention provides for the use of at least one statin or an analogue, derivative, metabolite, prodrug or salt thereof in the preparation of a medicament for the treatment of type A influenza infection in a subject.

In certain embodiments, the subject is a mammal, typically a human.

In certain embodiments, the Influenza A virus is of the subtype H3. In certain further embodiments, the subtype is H3N2.

In certain embodiments, the Influenza A virus is selected from the group comprising, but not limited to Influenza A virus of the subtype; H5, H7 or H9. In further certain embodiments the type A Influenza subtype is of the strain H5N1, H5N2, H9N2, H7N2 or H7N7.

A yet further aspect of the present invention provides for a pharmaceutical composition for the treatment and/or prophylaxis of type A influenza infection in a subject, wherein said composition comprises at least one statin or an analogue, derivative, metabolite, prodrug or salt thereof along with a pharmaceutically acceptable excipient, carrier or diluent.

Where the pharmaceutical composition comprises at least one pharmaceutical excipient, diluent or carrier, preferably these are selected according to the intended route of administration. Such materials should be non-toxic and should not interfere with the efficacy of the statin-containing composition.

In one embodiment the pharmaceutical composition is formulated in beta-hydroxycyclodextrin.

In certain embodiments a pharmaceutically acceptable excipient, carrier, buffer stabiliser may be used which is well known to those skilled in the art. Such materials should be non-toxic and should not interfere with the efficacy of the active ingredient.

The present inventor has also identified that the statin, statin metabolite or statin pro-drug compound or composition of the present invention may be administered along with a second anti-viral compound in order to provide a combined therapeutic which has utility in the treatment of viral disease. As such, in further aspects, the present invention extends to methods, pharmaceutical compounds and uses which comprise a combination therapeutic, the therapeutic comprising at least one anti-viral compound along with at least one statin, statin metabolite or statin pro-drug as defined hereinbefore. Such a combined medicament would have particular utility in the treatment of viral conditions which are highly infectious or pathogenic.

Accordingly, a yet further aspect of the present invention provides a method for the treatment and/or prophylaxis of type A influenza, the method comprising the steps of;
providing a composition comprising at least one statin or a pro-drug thereof,
administering a therapeutically effective amount of said composition to a subject in need of such treatment, and
further administering a therapeutically useful amount of at least one secondary anti-viral compound.

In certain embodiments, the statin may be at least one statin hydroxy acid salt selected from the group comprising, but not limited to; lovastatin hydroxy acid sodium salt, which may also be known as mevinolinic acid.

In certain embodiments, the statin pro-drug may be selected from the group consisting of, but not limited to: lovastatin, simvastatin, pravastatin, fluvastatin, cerivastatin and atorvastatin. The statin compound may further extend to an analogue, derivative or metabolite of lovastatin, simvastatin, pravastatin, fluvastatin, cerivastatin and atorvastatin.

In one embodiment the statin is pravastatin hydroxy acid salt, simvastatin hydroxy acid salt, or mevastatin hydroxy acid salt.

In one embodiment, the anti-viral compound is administered along with the statin or statin pro-drug composition, however, in further embodiments, the secondary anti-viral compound may be administered before or after the statin or statin pro-drug composition has been administered.

In preferred embodiments, the secondary anti-viral compound may be selected from at least one of the group comprising, but not limited to, ribavirin, amantadine, rimantadine, oseltamivir (TAMIFLU™) and zanamivir.

According to a yet further aspect of the present invention there is provided the use of statin or statin pro-drug and at least one anti-viral compound in the preparation of a combined medicament for the treatment or prevention of infection with type A Influenza.

In certain embodiments, the secondary anti-viral compound may be selected from the group comprising, but limited to, ribavirin, amantadine, rimantadine, oseltamivir (TAMIFLU™) or zanamivir.

In certain further embodiments, the secondary anti-viral compound is a vaccine compositions which, following administration to a subject, induces a protective immune response, typically a long-term protective immune response. Said vaccine composition may be further administered along with a suitable adjuvant.

In certain embodiments, the secondary anti-viral compound is administered simultaneously with the statin containing composition of the invention. In further embodiments, the anti-viral compound is administered sequentially with the statin containing composition, that is the anti-viral compound is administered before or after the statin containing composition is administered.

A yet further aspect of the present invention provides for a pharmaceutical composition for the treatment of type A influenza infection, said composition comprising at least one statin or a pro-drug thereof, and an anti-viral compound along with a pharmaceutically acceptable excipient, carrier or diluent.

In a certain embodiment, the pharmaceutical composition is formulated in beta-hydroxycyclodextrin.

Statins are not only effective in at least in part inhibiting infection by type A influenza virus, but also in reducing the speed with which the virus spreads to uninfected cells. In one embodiment a statin or a metabolite or precursor thereof is used for the preparation of a medicament for the treatment of an individual infected with type A influenza or an individual at risk of becoming infected with a type A influenza infection. Since statin or precursors thereof act on the level of infectivity they work synergistically with anti-viral compounds that act to prevent viral infection and spread by other means.

The present inventor has further identified that the statin compounds and hydroxy acid salts thereof have utility as a treatment and/or prophylactic for the prevention of infection with hepatitis virus, in particular hepatitis C.

Accordingly in a yet further aspect of the present invention provides a method for the treatment and/or prophylaxis of hepatitis C virus, the method comprising the steps of;
providing a composition comprising at least one statin or an analogue, derivative, metabolite, prodrug or salt thereof,
administering a therapeutically effective amount of said composition to a subject in need of treatment.

In one embodiment the method further comprises the steps of further administering a therapeutically effective amount of a suitable secondary anti-viral compound. The suitable secondary anti-viral compound may be administered sequentially or subsequently to the administration of the statin containing composition.

In certain embodiments, the subject is a mammal, typically a human.

In a further aspect of the present invention, there is provided the use of a statin or an analogue, derivative, metabolite, prodrug or salt thereof and an anti-viral compound in the preparation of a medicament for the prevention of infection with hepatitis virus. In one embodiment the hepatitis virus is hepatitis C.

A yet further aspect of the present invention provides for a pharmaceutical composition for the prophylaxis of human infection with a hepatitis C infection, wherein said composition comprises at least one statin or a pro-drug thereof along with a pharmaceutically acceptable excipient, carrier or diluent.

The invention further extends to methods, pharmaceutical compositions and uses for the prophylaxis and/or treatment of hepatitis A virus using the statin compounds or analogues, derivatives, metabolites, prodrugs or salts thereof.

### Synthesis of Statin Compounds

The active compounds of this disclosure, i.e. statin hydroxy acid salt, may be synthesized or isolated from natural sources such as red yeast rice as a source material. Mevinolinic acid (lovastatin hydroxy acid salt) is a known constituent of red yeast rice.

### Treatment/Therapy

As used herein, the term “therapeutically effective amount” means the amount of a compound or composition which is required to reduce the severity of and/or ameliorate influenza A viral infection, or at least one symptom thereof, or which serves to prevent the progression of influenza A infection or the development of one or more of the symptoms associated with mucositis.

As used herein, the term “prophylactically effective amount” relates to the amount of a composition which is required to prevent the initial onset, progression or recurrence of influenza A viral infection or at least one symptom thereof in a subject following the administration of the compounds of the present invention.

As used herein, the term “treatment” and associated terms such as “treat” and “treating” means the reduction of the progression, severity and/or duration of influenza A viral infection or the amelioration of at least one of the symptoms thereof, wherein said reduction or amelioration results from the administration of at least one of the compositions of the present invention. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition.

Accordingly, therapeutic and prophylactic treatments include amelioration of the symptoms of a particular condition or preventing or otherwise reducing the risk of developing a particular condition. The term “prophylactic” may be considered as reducing the severity of influenza A viral infection. “Therapeutic” may also reduce the severity of an existing condition.

As used herein, the term “subject” refers to an animal, preferably a mammal and in particular a human. In a particular embodiment, the subject is a mammal, in particular a human, who has been, or may be at risk of infection from influenza type A infection. The term “subject” is interchangeable with the term “patient” as used herein.

Accordingly, therapeutic and prophylactic treatment includes amelioration of the symptoms of a particular condition or preventing or otherwise reducing the risk of developing a particular condition. The term “prophylactic” may be considered as reducing the severity or the onset of a particular condition. “Therapeutic” may also reduce the severity of an existing condition.

### Administration

The statins or precursors thereof of the invention such may be administered alone but will preferably be administered as part of a pharmaceutical composition, which will generally also comprise a suitable pharmaceutical excipient, diluent or carrier which would be selected depending on the intended route of administration.

The statin products of the invention may be administered to a patient in need of treatment via any suitable route. The precise dose will depend upon a number of factors, including the precise nature of the form of the statin to be administered.

Routes of administration may include, but are not limited to; parenterally (including subcutaneous, intramuscular, intravenous, by means of, for example a drip patch), some further suitable routes of administration include (but are not limited to) oral, rectal, nasal, topical (including buccal and sublingual), infusion, vaginal, intradermal, intraperitoneally, intracranially, intrathecal and epidural administration or administration via oral or nasal inhalation, by means of, for example a nebuliser or inhaler, or by an implant.

In preferred embodiments, the composition is deliverable as an injectable composition, is administered orally, or is administered to the lungs as an aerosol via oral or nasal inhalation.

For administration via the oral or nasal inhalation routes, preferably the active ingredient will be in a suitable pharmaceutical formulation and may be delivered using a mechanical form including, but not restricted to an inhaler or nebuliser device. Further, where the oral or nasal inhalation routes are used, administration is by a SPAG (small particulate aerosol generator) may be used.

For intravenous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, Lactated Ringer's injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The composition may also be administered via microspheres, liposomes, other microparticulate delivery systems or sustained release formulations placed in certain tissues including blood.

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition (Dec. 15, 2000) ISBN 0-912734-04-3 and Pharmaceutical Dosage Forms and Drug Delivery Systems; Ansel, H. C. et al. 7th Edition ISBN 0-683305-72-7 the entire disclosures of which is herein incorporated by reference.

### Dose

The composition of the invention is preferably administered to an individual in a “therapeutically effective amount” or a “prophylactically effective amount” as defined hereinbefore. The actual amount administered in order to achieve these effects, as well as the rate and time-course of administration will depend on, and can be determined with due reference to, the nature and severity of the condition which is being treated, as well as factors such as the age, sex, weight of the patient to be treated and the route of administration. Toxicity and efficacy of the compositions can be determined by standard pharmaceutical procedures.

Unless otherwise defined, all technical and scientific terms used herein have the meaning commonly understood by a person who is skilled in the art in the field of the present invention.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

As used herein, terms such as “a”, “an” and “the” include singular and plural referents unless the context clearly demands otherwise. Thus, for example, reference to “an active agent” or “a pharmacologically active agent” includes a single active agent as well a two or more different active agents in combination, while references to “a carrier” includes mixtures of two or more carriers as well as a single carrier, and the like.

The present invention will now be described with reference to the following examples which are provided for the purpose of illustration and are not intended to be construed as being limiting on the present invention.

### BRIEF DESCRIPTION OF THE DRAWINGS

FIG. 1 shows a graph illustrating the level of viral inhibition against an increasing concentration of Mevinolinic acid in accordance with the results obtained in experiment 3(i),

FIG. 2, which shows a graph illustrating the level of viral inhibition against an increasing concentration of Mevinolinic acid in accordance with the results obtained in experiment 3 (ii),

FIG. 3 shows a graph illustrating the level of viral inhibition against an increasing concentration of Mevinolinic acid in accordance with the results obtained in experiment 3 (iii).

### DETAILED DESCRIPTION OF THE INVENTION

The present invention relates to methods of prophylactically treating a vertebrate including a human who may become exposed to an influenza virus of any strain by administering to said vertebrate or human at least one statin hydroxy acid salt (e.g. lovastatin hydroxy acid sodium salt also known by the name mevinolinic acid).

This is unexpected and significant in that it makes available for the first time a chemical entity that if administered can prevent infection with an influenza virus and its mode of action does not appear to rely on the inhibition of any specific enzyme of the influenza virus as strains resistant to the two known anti-influenza compounds also are prevented from establishing effective infection by the specified compounds of this patent.

The methods, compounds and uses of the present invention can be used for the prophylaxis and/or treatment of Influenza A virus of any subtype. In certain embodiments, the Influenza A virus is of the subtype H3. In certain further embodiments, the subtype is H3N2.

In certain embodiments, the Influenza A virus is selected from the group comprising, but not limited to Influenza A virus of the subtype; H5, H7 or H9. In further certain embodiments the type A Influenza subtype is of the strain H5N1, H5N2, H9N2, H7N2 or H7N7.

### Salts and Solvates

The active compounds disclosed herein can, as noted above, can be prepared in the form of their pharmaceutically acceptable salts. Pharmaceutically acceptable salts are salts that retain the desired biological activity of the parent compound and do not impart undesired toxicological effects. Examples of pharmaceutically acceptable salts are discussed in Berge et al., 1977, “Pharmaceutically Acceptable Salts,” J. Pharm. ScL, Vol. 66, pp. 1-19.

The active compounds disclosed may also be prepared in the form of their solvates. The term “solvate” is used herein in the conventional sense to refer to a complex of solute (e.g., active compound, salt of active compound) and solvent. If the solvent is water, the solvate may be conveniently referred to as a hydrate, for example, a hemihydrate, monohydrate, dihydrate, trihydrate, tetrahydrate, and the like.

### Prodrugs

The invention further extends to prodrugs of the compounds of the present invention. A prodrug of any of the compounds can be made using well known pharmacological techniques.

### Homologues and Analogues

The present invention is further intended to encompass, in addition to the use of the above listed compounds, the use of homologues and analogues of such compounds. In this context, homologues are molecules having substantial structural similarities to the above-described compounds and analogues are molecules having substantial biological similarities regardless of structural similarities.

The invention further provides kits for carrying out the therapeutic regimens of the invention. Such kits may comprise, in one or more containers, therapeutically or prophylactically effective amounts of the compositions of the invention in a pharmaceutically acceptable form. Such kits may further include instructions for the use of the compositions of the invention, or for the performance of the methods of the invention, or may provide further information to provide a physician with information appropriate to treating mucositis.

### EXAMPLES

### Example 1

### Prophylactic Treatment of Cotton Rats (S. hispidus) Against Infection with Influenza A/Wuhan (H3N2)

### Materials and Methods:

Animals were treated using the following protocol:

Day 0-70 young adult cotton rats (S. hispudus) 6-8 weeks old were divided into 7 groups of 10 animals each. Each animal was pre-bled and ear tagged and all animals were treated with the preparations as shown in Table 1.

TABLE 1

| Group | Treatment |
|---|---|
| A | Untreated/Uninfected |
| B | Mock treated/Wuhan 10⁷ |
| C | Comp. dose 1/Wuhan 10⁷ |
| D | Comp. dose 2/Wuhan 10⁷ |
| E | Comp. dose 3/Wuhan 10⁷ |
| F | Zanamivir 0.5 mg/ml/Wuhan 10⁷ |
| G | Oseltamivir 1.75 mg/ml/Wuhan 10⁷ |

Each mouse in groups C to G was intranasally inoculated with 0.1 ml of the preparation indicated in groups C to G. Group H mice were subject to gastric gavage with 0.2 ml.

Day 2—Intranasal inoculation of groups C-G and gastric gavage of group H was repeated.

Day 3—Intranasal inoculation of groups C-F and gastric gavage of group H was repeated.

Day 4—Groups B-H were infected intranasally with Influenza A/Wuhan at 10⁷ TCID50 at 0.1 ml volume.

Day 5—5 animals from each group were euthanized and the lung bloc was removed. Trisection for viral quantitation, histopathology, and cytokine/chemokine analysis (TNF-alpha and IFN-alpha) was performed.

Day 6—The procedure performed on day 5 was repeated for the remaining animals in all groups.

### Example 2

### Prophylactic Treatment of Cotton Rats (S. hispidus) Against Infection with Influenza A with Wuhan 10⁷

### Materials and Methods:

Day 0-70 young adult cotton rats (S. hispudus) which were 6-8 weeks old were divided into 7 groups of 10 animals. All animals were pre-bled and ear tagged. Animals in groups B to H were treated intranasally with 0.1 ml of Influenza A/Wuhan (H3N2). The specific treatments are shown below in Table 2.

TABLE 2

| Group | Treatment |
|---|---|
| A | Untreated/Uninfected |
| B | Wuhan 10⁷/Mock |
| C | Wuhan 10⁷/Comp. dose 1 |
| D | Wuhan 10⁷/Comp. dose 2 |
| E | Wuhan 10^{7/}Comp. dose 3 |
| F | Wuhan 10⁷/Zanamivir 0.5 mg/ml |
| G | Wuhan 10⁷/Oseltamivir 1.75 mg/ml |

Day 2—Groups C to G were treated with 0.1 ml of indicated preparations intranasally. Group H was treated with 0.2 ml via gastric gavage.

Day 3—The procedure of day 2 was repeated.

Day 4—The procedure of day 2 was repeated.

Day 5—5 animals from each group were euthanized. Subsequent removal of the lung bloc was performed. Trisection for viral quantitation, histopathology, and cytokine/chemokine analysis (TNF-alpha and IFN-alpha) was performed.

Day 6—The procedure used on day 5 was repeated for all remaining groups.

### Example 3

### Evaluation of the Inhibitory Effect of Mevinolinic Acid (Lovastatin Hydroxy Acid Sodium Salt) on Replication of Influenza a H5N1 In-Vitro

### Materials and Methods:

The canine kidney derived cell line (Mardin Darby Canine Kidney, MDCK) that have been used widely for isolating influenza virus, and determining anti-influenza effects was used as a host cell line.

The H5N1 virus isolate was confirmed to be Influenza A H5N1 by the WHO reference centre. The full genome is available from the GenBank database (URL-http://www.ncbi.nlm.nih.gov/Genbank/index.html). This virus was isolated from a human infected with H5N1 in Hong Kong in 1997. The virus stock used was the third passage from the original isolate.

RIBAVIRIN™ which is known to have an in-vitro effect on influenza A was used as positive control. RIBAVIRIN™ was used at concentrations that have been determined to produce inhibitory effects on influenza H5N1 infection for the corresponding testing system used herein.

Two commonly employed methods were used in parallel for testing the compound mevinolinic acid. The first method was a virus yield reduction assay which was conducted using two approaches.

In the first approach, known as “without adsorption”, the virus inoculum was mixed with the test compound at various concentrations. After mixing and incubating for 30 minutes, the virus-drug mixtures were added to testing wells containing MDCK cells in triplicates. The amount of viruses produced after 24 hour incubation at 37° C. were then determined by a standard plaque assay which quantified the amount of infectious virus particles present.

In the second approach, known as “with adsorption”, the procedures were essentially the same with exception that the virus inoculum was added to the MDCK cells and incubated for one hour before the drug was added. This later approach allows the viruses to enter the MDCK cells before the drug has any chance to take action. Therefore, the “with adsorption” approach serves as a confirmatory assay for compounds targeting virus life cycle steps following virus entry.

The second anti-viral assay used in this study was a standard plaque reduction assay. This technique is commonly used to ascertain antiviral effects. The virus inoculum was added to MDCK cells, which were then overlayed with a semi-solid agar containing various drug concentrations. The semi-solid agar limits the spread of viruses released from infected cells. Therefore, each plaque observed after 24 hours of incubation represents one infectious unit of virus.

Thus, in this example, three independent anti-viral assays were conducted to verify the inhibitory effect of Mevinolinic acid on influenza A H5N1.

### Results:

(i) Virus Yield Reduction Assay—without Adsorption Technique

In these experiments, Mevinolinic acid added simultaneously with virus inoculum.

The virus tested was Influenza A H5N1 (isolated in 1997 from human). The virus inoculum was 1×10⁻³ pfu/cell. The results are shown in Table 3 below.

TABLE 3

| Drug concentration | Number of | Cytotoxicity in virus yield |
|---|---|---|
| (μg/mL) | plaques | reduction assay |
| 100 | 0 | Yes |
| 50 | 0 | Yes |
| 25 | 96000 | No |
| 12.5 | 151000 | No |
| 6.25 | 166000 | No |
| 3.125 | 228000 | No |
| 1.5625 | 220000 | No |
| 0.78 | 239000 | No |
| 0.39 | 244000 | No |
| Positive Control, | 11000 | No |
| RIBAVIRIB (10 μg/mL) | | |
| Virus Control (No drug) | 240000 | No |

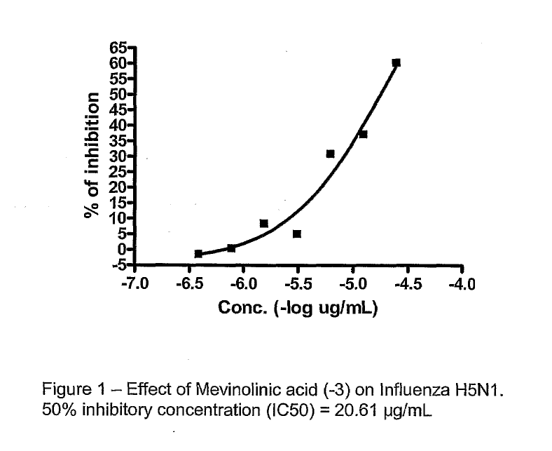

The results are further illustrated in FIG. 1, which shows a graph illustrating the level of viral inhibition against an increasing concentration of mevinolinic acid, in accordance with the results obtained in this experiment.

(ii) Virus Yield Reduction Assay—with Adsorption Technique

In these experiments, mevinolinic acid was added one hour after virus adsorption.

The virus tested was Influenza A H5N1 (strain isolated in 1997 from human). The virus inoculum was 1×10⁻³ pfu/cell. The results are shown in Table 4 below.

TABLE 4

| Drug conc. (μg/mL) | No. of plaque | Cytotoxicity |
|---|---|---|
| 50 | 0 | +/− |
| 25 | 1000 | No |
| 12.5 | 27000 | No |
| 6.25 | 30000 | No |
| 3.125 | 34000 | No |
| 1.5625 | 34000 | No |
| 0.78 | 38000 | No |
| 0.39 | 40000 | No |
| 0.19 | 41000 | No |
| 0.097 | 43000 | No |
| Positive Control, | 0 | No |
| RIBAVIRIN (5 μg/mL) | | |
| Virus Control (No drug) | 54000 | No |

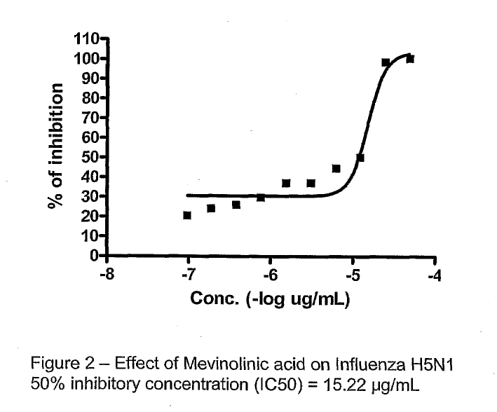

The results are further shown in FIG. 2, which shows a graph illustrating the level of viral inhibition against an increasing concentration of Mevinolinic acid in accordance with the results obtained in this experiment.

(iii) Plaque Reduction Assay

The virus tested was Influenza A H5N1 (isolated in 1997 from human). The virus inoculum was adjusted to produce around 200 plaques per well. The results are shown in Table 5 below.

TABLE 5

| | No. of | No. of | No. of | |
|---|---|---|---|---|
| Drug conc. | plaque | plaque | plaque | |
| (μg/mL) | 1st well | 2nd well | 3rd well | Cytotoxicity |
| 25 | 113 | 100 | 110 | No |
| 12.5 | 192 | 181 | 180 | No |
| 6.25 | 223 | 215 | 222 | No |
| 3.125 | 230 | 226 | 221 | No |
| 1.5625 | 240 | 246 | 243 | No |
| 0.78 | 257 | 264 | 258 | No |
| Positive Control, | 134 | 131 | 132 | No |
| RIBAVIRIN | | | | |
| (5 μg/mL) | | | | |
| Virus Control | 260 | 263 | 269 | No |
| (no drug) | | | | |

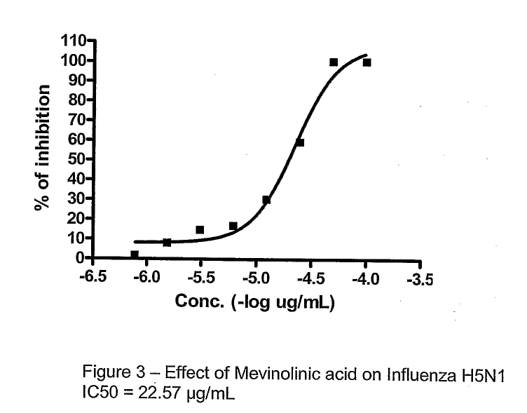

FIG. 3 shows a graph illustrating the level of viral inhibition against an increasing concentration of mevinolinic acid in accordance with the results obtained in this experiment.

### SUMMARY

The results of these experiments indicate that mevinolinic acid has an inhibitory effect on the replication of influenza A H5N1 virus as demonstrated by in-vitro analysis of influenza infection of Mardin Darby Canine Kidney (MDCK) cells. When the virus yield reduction method was used, Mevinolinic acid archived a 50% reduction in virus replication, i.e. inhibitory concentration 50 (IC₅₀), at a drug concentration of 20.61 μg/mL when the compound was added simultaneously with the virus inoculum, the IC₅₀ was 15.22 μg/mL when mevinolinic acid was added after a one hour virus adsorption. This suggests the compound mevinolinic acid acted at a stage after viral entry into the host cell. Mevinolinic acid also demonstrated an inhibitory effect with IC₅₀ of 22.57 μg/mL when a further experiment, using a plaque reduction assay, was performed.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A method for the treatment and/or prophylaxis of Influenza type A infection, the method comprising the steps of:
providing a composition comprising at least one statin or an analogue, derivative, metabolite, prodrug or a pharmaceutically acceptable salt thereof, and
administering a therapeutically effective amount of said composition to a subject in need of such treatment.

2. The method as claimed in claim 1 wherein the statin is a statin hydroxy acid salt.

3. The method as claimed in claim 1 wherein the statin is lovastatin hydroxy acid sodium salt or a prodrug or metabolite thereof.

4. The method as claimed in claim 1 wherein the statin is selected from the group consisting of simvastatin, lovastatin, pravastatin, fluvastatin, cerivastatin and atorvastatin.

5. The method as claimed in claim 1 further comprising the step of administering a secondary anti-viral compound to the subject.

6. The method as claimed in claim 5 wherein the secondary anti-viral compound is selected from the group consisting of ribavirin, amantadine, rimantadine, oseltamivir or zanamivir.

7. (canceled)

8. (canceled)

9. (canceled)

10. (canceled)

11. A pharmaceutical composition for the treatment of type A influenza infection, wherein said composition comprises at least one statin or an analogue, derivative, metabolite, prodrug or a pharmaceutically acceptable salt thereof along with a pharmaceutically acceptable excipient, carrier or diluent.

12. The pharmaceutical composition as claimed in claim 11 wherein the at least one statin is selected from the group consisting of simvastatin, lovastatin, pravastatin, fluvastatin, cerivastatin and atorvastatin.

13. The pharmaceutical composition as claimed in claim 11, wherein the composition further comprises at least one secondary anti-viral compound.

14. The pharmaceutical composition as claimed in claim 13 wherein the at least one secondary anti-viral compound is selected from the group consisting of ribavirin, amantadine, rimantadine, oseltamivir or zanamivir.

15. The pharmaceutical composition as claimed in claim 11 wherein the statin is formulated in beta-hydroxycyclodextrin.

16. The pharmaceutical composition as claimed in claim 11 wherein the statin is a statin hydroxy acid salt.

17. The pharmaceutical composition as claimed in claim 11 wherein the statin is lovastatin hydroxy acid sodium salt.

18. The method as claimed in claim 1 wherein the influenza type A infection is infection by the subtype H3, H5, H7 or H9.

19. The method as claimed in claim 18 wherein the influenza type A infection is infection by the strain H5N1.
